(19) United States
(12) Patent Application Publication
(10) Pub. No.: US 2023/0184759 A1
(43) Pub. Date: Jun. 15, 2023

(54) IN VITRO METHOD FOR DETECTION OF INFECTIONS CAUSED BY PSEUDOMONAS AERUGINOSA

(71) Applicant: CONSEJO SUPERIOR DE INVESTIGACIONES CIENTIFICAS (CSIC), Madrid (ES)

(72) Inventors: M. Pilar MARCO COLÁS, Madrid (ES); Enrique José MONTAGUT CAÑETE, Madrid (ES)

(21) Appl. No.: 17/915,319

(22) PCT Filed: Mar. 26, 2021
(86) PCT No.: PCT/EP2021/057909

§ 371 Date: Sep. 28, 2022

(30) Foreign Application Priority Data
Mar. 31, 2020 (EP) 20382256.4

(51) Int. Cl. G01N 33/569; G01N 33/535; C07D 215/233; A61K 39/385; C07K 16/12

(52) U.S. Cl. CPC G01N 33/56911; G01N 33/535; C07D 215/233; A61K 39/385; C07K 16/1214; G01N 2333/21; G01N 2458/15; A61K 2039/6012

(57) ABSTRACT

In vitro method for detection of infections caused by Pseudomonas aeruginosa. The present invention relates to compounds of general Formula (I) and to their use as haptens. Moreover, the present invention also refers to conjugates comprising the haptens of the invention and to their use for obtaining antibodies. Finally, the invention also relates to an in vitro method for the detection of infections caused by Pseudomonas aeruginosa by means of the identification and/or quantification of the main signaling molecules from the pqs quorum sensing system.

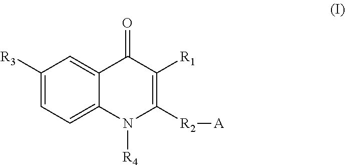

### FIELD OF THE INVENTION

The present invention pertains to the medical field. Particularly, the present invention relates to compounds of general Formula I and to their use as haptens. Moreover, the present invention also refers to conjugates comprising the haptens of the invention and to their use for obtaining antibodies. Finally, the invention also relates to an in vitro method for the detection of infections caused by Pseudomonas aeruginosa by means of the identification and/or quantification of the main signaling molecules from the pqs quorum sensing system (pqs QS system).

### PRIOR ART

Pseudomonas aeruginosa is a gram-negative, ubiquitous bacterium cause of a broad spectrum of human diseases such as pneumonia, septicemia and other life-threatening acute and chronic infections. This bacterium belongs to the group of so-called ESKAPE pathogens, a classification of multidrug resistant “superbugs” based on prevalence, 10-years trend of resistance, transmissibility, treatability and preventability in hospital and community settings. This group of microorganisms represents a serious global health threat for which traditional therapeutic options have become limited. Therefore, there is an increasing urgent need of finding novel strategies to deal with this new generation of resistant pathogens. The correct detection and fast identification of the responsible pathogens in these infections is crucial for an adequate treatment. On the other hand, the lack of diagnostic methods capable of providing reliable and fast results has led to the prescription and misuse of broad-spectra antibiotics, contributing to the generation of resistance.

Current methods are based on culture techniques, which can take up to 72 h in order to obtain conclusive results, commonly triggered by a deficit in both sensitivity and specificity of the technique itself. Molecular detection tools have emerged as an interesting approach to overcome the lack of sensitivity and rapidity of the actual methods. Namely, MALDI-TOF analysis or PCR methods have been successfully developed for detection of different bacteria in either isolates or clinical samples. However, these methods normally require specific equipment, highly qualified personnel, tedious extractions and/or purification steps. Immunochemical-based techniques appear as an interesting alternative and provide great versatility through their application in both optical and electrochemical sensors.

Therefore, there is a need in the state of the art to develop methodologies alternative to those described in the state of the art for the detection of infections caused by Pseudomonas aeruginosa in biological samples, in particular by immunochemical methods.

The present invention is directed to solve this problem by providing for first time, an immunochemical assay for the identification and/or quantification of molecules from pqs quorum sensing system of Pseudomonas aeruginosa, allowing their evaluation as biomarkers of disease for diagnostic purposes.

### DESCRIPTION OF THE INVENTION

### Description of the Invention

The present invention relates to compounds of general Formula I and to their use as haptens. Moreover, the present invention also refers to conjugates comprising the haptens of the invention and to their use for obtaining antibodies. On the other hand, the invention also relates to an in vitro method for the detection of infections caused by Pseudomonas aeruginosa by means of the identification and/or quantification of molecules from the pqs QS system, particularly the molecules: 2-heptyl-4-quinolone (HHQ), 2-heptyl-3-hydroxy-4-quinolone (PQS), and/or 2-heptyl-4-quinolone N-oxide (HQNO), using said antibodies and conjugates. The molecules HHQ, PQS and HQNO are characterized by the following formula:

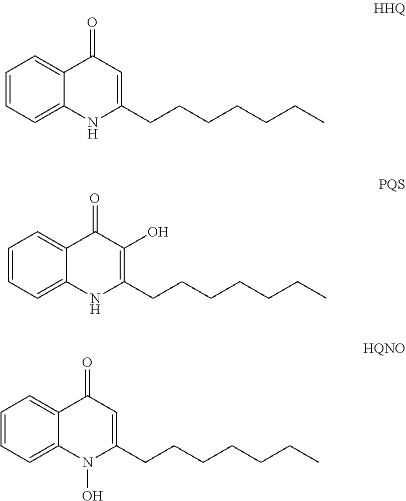 [SMILES: CCCCCCCc1cc(=O)c2ccccc2[nH]1 ; CCCCCCCc1[nH]c2ccccc2c(=O)c1O ; CCCCCCCc1cc(=O)c2ccccc2n1O]

Particularly, the first embodiment of the present invention refers to a compound characterized by the Formula I,

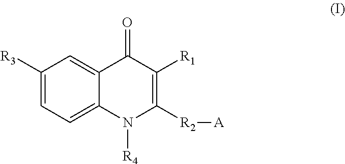

wherein: R₁ is selected among H, OH or COO—R; R is selected among H, C1-C4 alkyl or NH₂; R₂ is selected among C3-C15 alkyl, C3-C15 alkenyl or C3-C15 alkynyl; A is selected among H, COOH, NH₂ or SH; R₃ is selected among C2-C10 alkyl or (CH₂)ₘ—R₅; m is a whole number between 1 and 5; R₁ is selected among H or OH; and R₅ is selected among COOH, SH, NH₂, OH or PEG; or any combination thereof. In a particularly preferred embodiment, the compounds characterized by the Formula I are:

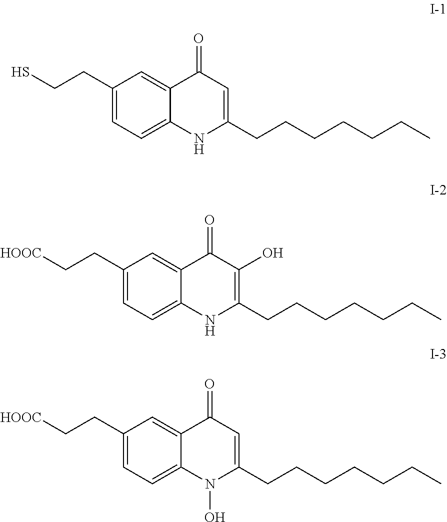 [SMILES: CCCCCCCc1cc(=O)c2cc(CCS)ccc2[nH]1 ; CCCCCCCc1[nH]c2ccc(CCC(=O)O)cc2c(=O)c1O ; CCCCCCCc1cc(=O)c2cc(CCC(=O)O)ccc2n1O]

The second embodiment of the present invention refers to the use of at least a compound characterized by the Formula I as a hapten. So, the haptens are structurally related to HHQ, PQS and HQNO secreted by the Gram-negative bacteria P. aeruginosa (hereinafter the analytes), for the production of specific antibodies against these analytes. In particular, with the antibodies produced, a diagnostic tool has been developed which allows the detection and/or quantification of HHQ, PQS and HQNO in biological samples of patients who may have these bacteria.

Haptens of general Formula I may be prepared following different methods known by a person skilled in the field of organic synthesis, in particular they may be synthesised following the strategy previously described by Reen et al. (Org Biomol Chem 2012, 10, 8903) but including a functionalized spacer arm susceptible of being conjugated to a carrier protein. In order to maximize the exposure of the most important epitopes, the spacer arm was placed at the C-6 position of the quinolone structure (see Scheme 1)

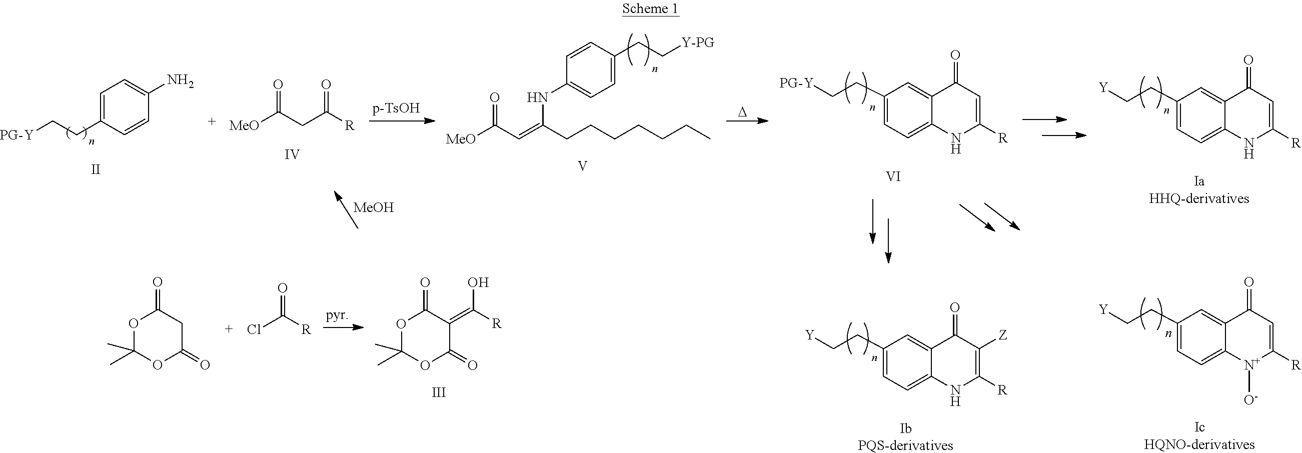

Therefore, the starting aniline derivative is protected adequately to obtain aniline II. In parallel, Meldrum's acid is made to react with an alkanoyl halide to obtain a derivative III that after methanolysis, gives the desired β-ketoester IV. Afterwards, the condensation reaction between the aniline derivative and β-ketoester is carried out in the presence of a catalytic amount of p-toluene sulfonic acid to obtain enamine V. Finally, a thermic cyclization in diphenyl ether is performed to obtain the desired 4-quinolone structure VI.

To obtain HHQ type haptens an adequate desprotection of the protected functional group at the spacer arm in VI yields haptens Ia derived of HHQ structure.

The synthetic strategy to obtain PQS type haptens (Ib) is carried out following the same procedure as described before for HHQ-derivatives. Once obtained 4-quinolone VI, functionalization of C-3 is carried out by Duff reaction, using hexamine as formyl carbon source using conditions described by Pesci et al. (PNAS 1999, 99, 11229). Transposition to obtain hydroxyl group is performed by Dakin oxidation, which starts with hydrogen peroxide nucleophilic addition followed by hydroxide elimination (Scheme 2).

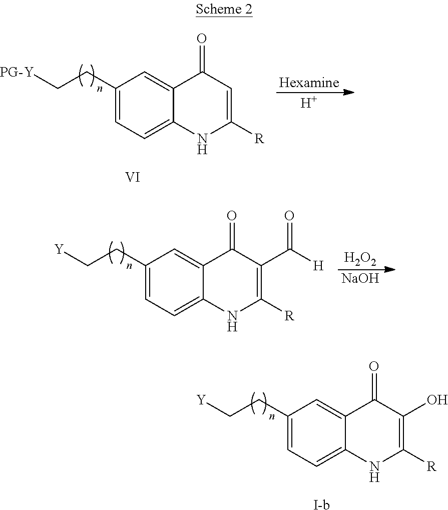

HQNO type haptens (Ic) can be obtained following the experimental procedure described by Woschek et al. (Synthesis 2007(10): 1517-1522 Woschek, A.; Mahout, M.; Mereiter, K.; Hammerschmidt, F.; Hammerschmidt, F Synthesis of 2-Heptyl-1-hydroxy-4 (1H)-quinolone—Unexpected Rearrangement of 4-(Alkoxycarbonyloxy)quinoline N-Oxides to 1-(Alkoxycarbonyloxy)-4(1H)-quinolones. Synthesis 2007, 2007 (10), 1517-1522.) (Scheme 3). Thus, once obtained 4-quinolone VI, as described before, the high nucleophilicity shown by position C-3 and the carbonyl at C-4 is avoided by blocking the corresponding tautomer through protection reaction. For this purpose, C-4 hydroxyl group can be protected using Boc anhydride or any appropriate protecting group followed by oxidation of nitrogen with m-CPBA to achieve the characteristic N-oxide compound. Finally, the desired hapten Ic is obtained by basic deprotection of functional groups.

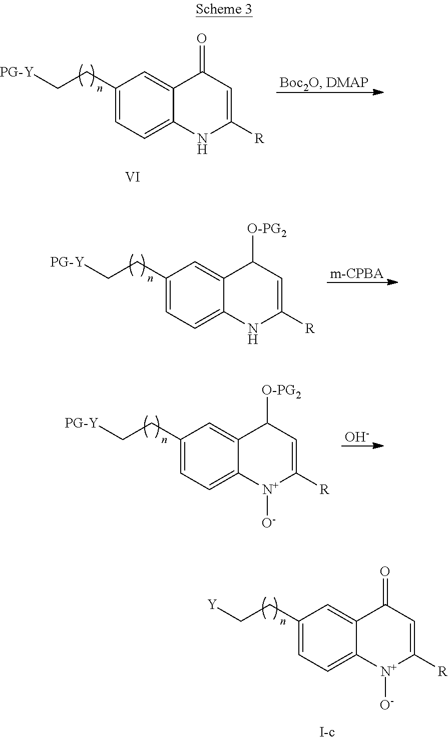

The third embodiment of the present invention refers to a conjugate comprising at least a hapten according to Formula I in combination with a second component which confers antigenicity to the conjugate. In a preferred embodiment, the second component is a carrier protein, or a fragment thereof, preferably selected from the group comprising: horseshoe crab hemocyanin (HCH), bovine serum albumine (BSA) or keyhole limpet hemocyanin (KLH). In a preferred embodiment, the conjugate is formed by a covalent bond between the R₃ of Formula I and the carrier protein.

In a preferred embodiment the conjugates of the invention are listed in Table 1 below.

TABLE 1
Conjugates of the invention

| | |
|---|---|
| I-4 | 2-((2-(2-heptyl-4-oxo-1,4-dihydroquinolin-6- |
| | yl)ethyl)thio)acetamide-HCH |
| I-5 | 2-((2-(2-heptyl-4-oxo-1,4-dihydroquinolin-6- |
| | yl)ethyl)thio)acetamide-BSA |
| I-6 | 3-(2-heptyl-3-hydroxy-4-oxo-1,4-dihydroquinolin- |
| | 6-yl)propanamide-KLH |
| I-7 | 3-(2-heptyl-3-hydroxy-4-oxo-1,4-dihydroquinolin- |
| | 6-yl)propanamide-BSA |
| I-8 | 3-(2-heptyl-1-hydroxy-4-oxo-1,4-dihydroquinolin- |
| | 6-yl)propanamide-KLH |
| I-9 | 3-(2-heptyl-1-hydroxy-4-oxo-1,4-dihydroquinolin- |
| | 6-yl)propanamide-BSA |

The fourth embodiment of the present invention refers to a method for producing a conjugate as defined above, which comprises creating a covalent bond, directly or through a cross-linking agent, between the carrier protein and at least a hapten of Formula I. In a preferred embodiment, the conjugate is formed by a covalent bond between the R₃ of Formula I and the carrier protein.

Conjugates may be prepared according to various methods known to anyone skilled in the field of organic and immunochemical synthesis, particularly, general procedures that are shown in the following schemes. Starting materials for preparative methods are commercially available or can be prepared using the methods described in the literature.

In general, in the conjugates of the invention between a hapten and a protein, the proteins are bonded to the hapten covalently by means of the amino acids accessible on their surface, preferably those amino acids with nucleophile-type side chains. The reactive amino acid of the proteins is selected from the list comprising, but without being limited to, cysteine, serine, tyrosine and lysine; it is preferably lysine. The processes to achieve the conjugation of haptens to other carrier molecules depend on the functional group present in the hapten molecule in question. It must also consider the stability and solubility of the hapten. Therefore, given the large variety of haptens that exist, there is no common conjugation method.

In some methods, the hapten and the carrier protein are bound by a cross-linking agent.

For the protein cross-linking, the functional protein groups whereto to the cross-linking agents are targeted comprise amino groups, ε-amino groups of lysine, α-amino terminal groups, cysteine sulfhydryl groups (—SH or thiol groups), carbohydrate groups (in the case of glycoproteins) and carboxyl groups.

Cross-linking agents of proteins through amino groups, lysine ε-amino and terminal α-amino groups include, but without being limited to imidoesters and N-hydroxysuccinimide esters (NETS-esters).

Cross-linking agents of proteins through sulfhydryl groups include, without being limited to, maleimides, haloacetyls (such as iodoacetyl) and pyridyl disulfide (pyridyldithiols).

Cross-linking agents of proteins through carbonyl groups (such as aldehydes or ketones) by oxidative treatment of the glycoprotein carbohydrates include, without being limited to, reagents comprising hydrazides (—NH—NH2-).

Cross-linking agents of proteins through carboxyl groups include, without being limited to, carbodiimides.

Some methods are shown here by way of illustration and not in a limiting sense, since other conjugation methods known by persons skilled in the art may be used.

Haptens with a thiol group can be covalently attached to a carrier protein (SI), which is activated with groups capable of reacting with said thiol group (see Scheme 4) by means of a cross-linking agent such as succinimidyl esters or with any other active ester, having in their structure reactive features with the thiol group and then react with the thiol hapten to obtain the corresponding conjugate.

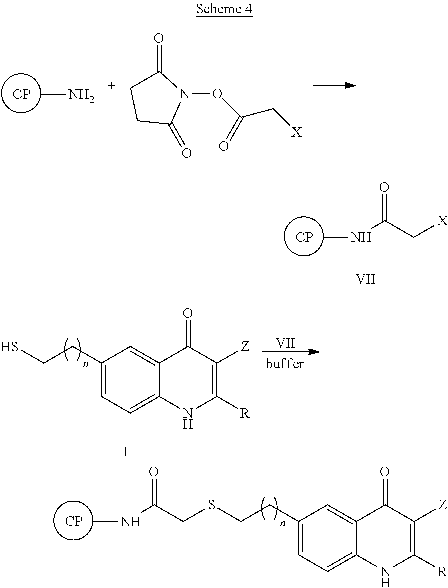

In the case of haptens with amine and carboxylic groups, they can be conjugated with the carrier protein, among others, using the mixed anhydride method, the carbodiimide method (CDI) or the N-hydroxysuccinimide ester method (NETS) (this latter also known as active ester method).

The fifth embodiment of the present invention refers to the use of a conjugate as defined above for producing antibodies. In a preferred embodiment, the conjugates used as immunogens for the production of antibodies are: I-4, I-6 and I-8.

The sixth embodiment of the present invention refers to an antibody characterized in that it specifically recognizes a conjugate of the invention, or to antiserum comprising said antibody. In a preferred embodiment, the antibodies are, for example, polyclonal antibodies or monoclonal antibodies, intact, or fragments thereof; and includes human, humanized and non-human origin antibodies.

The seventh embodiment of the present invention refers to an in vitro method for detecting and/or quantifying at least a quinolone selected from the group: HHQ, PQS and/or HQNO, in a biological sample, which comprises the use of an antibody or antiserum as defined above.

The antibodies of the invention are valid for their use in any type of immunochemical analysis configuration such as, for example, ELISA-type formats, lateral-flow immunoassay (LFIA,) or of strip, Western-blot, immunoturbidimetry or immunosensors. They are also useful for the preparation of immunoaffinity extraction systems, whether, although not being limited to, immunoaffinity columns or particles biofunctionalized with the antibody, or any other type of support which allows the anchoring of the antibody for the later use of the biohydrid material for the extraction by specific interactions with the antibody.

In a preferred embodiment, the method is carried out by an ELISA assay. The ELISA can be a direct ELISA, indirect ELISA, sandwich ELISA of competitive or non-competitive type. Preferably is a competitive indirect ELISA.

In a preferred embodiment, the method comprises: Immobilizing a conjugate as defined above on a solid support, eliminating the non-immobilized conjugate, adding the sample to be analysed and a first antibody defined above in the solid support of section and incubating, eliminating the first antibody not bound to the conjugate, adding a second antibody conjugated with a detectable labelling agent, said second antibody recognizing the first antibody and incubating, eliminating the second antibody not bound to the first antibody, and detecting and/or quantifying the complex obtained with a composition containing a chromogenic, fluorogenic and/or chemiluminescent indicator substrate. In a preferred embodiment, the sample is obtained from a subject who may have an infection caused by P. aeruginosa. In a preferred embodiment, the sample is selected from the group: sputum, bronchoaspirate (BAS), bronchoalveolar lavage (BAL), blood, serum and/or plasma.

In a preferred embodiment the immobilized conjugates, acting as competitor or coating antigens, are different that those used as immunogens.

In a preferred embodiment the indicator substrate is chromogenic, and the reaction is enzymatic.

The eight embodiment of the present invention refers to a kit for the detection and/or quantification of a quinolone selected from the group: HHQ, PQS and/or HQNO, characterized in that it comprises at least one antibody and a conjugate as defined above.

The ninth embodiment of the present invention refers to a method for treating an infection caused by P. aeruginosa which comprises a previous step wherein the infection is diagnosed following the method of the invention described above.

For the purpose of the present invention the following terms are defined:
The term “comprising” or “comprise” is meant including, but not limited to, whatever follows the words “comprising” or “comprise”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase” consisting of indicates that the listed elements are required or mandatory, and that no other elements may be present.
The term “antibody”, as used here in the present invention, relates to immunoglobulin molecules and immunologically active portions of immunoglobulin molecules, i.e. molecules containing an antigen fixing site which specifically binds (immunoreacts) with an antigen, such as, for example, a protein. There are 5 isotypes or main classes of immunoglobulins: immunoglobulin M (IgM), immunoglobulin D (IgD), immunoglobulin G (IgG), immunoglobulin A (IgA) and immunoglobulin E (IgE). The term “antibody” comprises any type of known antibody such as, but not being limited to, for example, polyclonal antibodies or monoclonal antibodies, intact, or fragments thereof; and includes human, humanized and non-human origin antibodies. In the context of this invention, the term antibody relates to the immunoglobulin that the animal or a hybrid cell has synthesized specifically against the conjugated hapten of the invention.
“Monoclonal antibodies” are homogenous populations of identical antibodies, produced by a hybrid cell product of the fusion of a clone of B lymphocytes descendent of a single and unique stem cell and a tumour plasma cell, which are directed against a single site or antigenic determinant. “Polyclonal antibodies” include heterogeneous populations of antibodies, which are directed against different antigenic determinants.
The term “antigen” refers to a molecule, such as a peptide, a carbohydrate, a glycolipid, a glycoprotein or a small molecule which is recognized and is bound to an antibody. The part of the antigen which is the target of the antibody bond corresponds to the antigenic determinant. In the context of the present invention, the antigen relates to a hapten according to the invention conjugated with a carrier protein, said conjugate being the one that is recognized and is bound to the specific antibodies obtained against the quinolone analytes of the invention.
The term “conjugate” relates in the present invention to the complex formed by the covalent bond of a hapten according to the invention and a second component which is selected from the group formed by a carrier protein or a fragment thereof which gives antigenicity, a detectable tag and a polymer or a support. Methods for producing hapten-carrier protein conjugates are known by a person skilled in the art.
The term “hapten”, as used in the present invention, relates to a molecule of low molecular weight, which by itself is not capable of generating an immune response in an animal and needs to be bound to a carrier molecule to generate an immune response. Therefore, the hapten is a small molecule of non-immunogenic character with the capacity of inducing the formation of antibodies when it is bound to a carrier molecule, in particular a carrier protein.
The term “carrier protein” or “transport protein” or “carrier”, in the context of the present invention, relates to a protein or to a fragment thereof which, on being bound to a hapten, is responsible that said hapten, in an animal organism, turns into an immunogen with the capacity of inducing antibody formation. In said conjugate the hapten is responsible for inducing the desired specificity in the immune response, and the carrier molecule is responsible for giving antigenicity to the hapten, i.e. the capacity of behaving as an antigen. Proteins useful as carrier molecules for this invention are proteins with a molecular mass greater than 10 kDa, preferably greater than 15 kDa. Examples of carrier proteins according to the invention include, without being limited to, horseshoe crab hemocyanin (HCH), keyhole limpet hemocyanin (KLH), serum albumin of various species such as bovine serum albumin (BSA), rabbit serum albumin (RSA), horseradish peroxidase (HRP), ovalbumin (OVA), conalbumin (CONA), thyroglobulin and fibrinogen, and fragments of said proteins which give antigenicity. Preferred carrier proteins according to the invention are horseshoe crab hemocyanin (HCH), bovine serum albumin (BSA) and keyhole limpet hemocyanin (KLH).
The term “immunochemical technique of analysis” is an immunochemical method of analysis wherein an antibody is used which specifically binds to an antigen. The immunochemical technique of analysis is characterized by the use of specific binding properties of a particular antibody to isolate, direct and/or quantify the antigen. The immunochemical techniques comprise, without being limited to, immunoassays such as ELISA (Enzyme-Linked Immunosorbent Assay), LFIA (Lateral-flow immunoassay) Western-blot, RIA (radioimmunoassay), competitive EIA (enzyme immunoassay), DAS-ELISA (Double Antibody Sandwich-ELISA), immunocytochemical techniques and immunohistochemical techniques, techniques based on the use of biomarker, biosensor or microarray biochips, which include specific antibodies or assays based on colloidal precipitation in formats such as “dipsticks”. Other immunochemical techniques include immunosensors whose transduction principle may be optical, electrochemical, piezoelectric, mass or thermometric. It also includes the immunosorbents or immunoaffinity extraction systems, which allow the selective extraction of an analyte within a complex mixture. These systems, usually of biohydrid materials, result from the stable union of the antibody to a solid support (polymer, inorganic material, metal particles, etc.), and which are used for the separation or extraction of the analyte from the rest of the matrix's components.
The term “immunogen” as used in the present invention, relates to a conjugate according to the invention capable of triggering an immune response. The term “antiserum” relates to a serum obtained after the immunization of an animal with an immunogen. The antiserum comprises specific antibodies of said immunogen generated after the immune response produced in the animal.
The term “sample”, as used in the present invention, relates to a sample to be analyzed by the method of the invention, susceptible of containing HHQ, PQS and/or HQNO as markers of infections caused by P. aeruginosa, which has been previously obtained from the subject under study (unless indicated otherwise). Illustrative, non-limiting examples of samples include both biological samples of tissues and body fluids, such as, for example, blood, serum, plasma, saliva, sputum, ear suppurations, bronchial washes, tissue exudates, etc. In a particular embodiment, said sample is sputum. In another particular embodiment, said sample is plasma. In another particular embodiment, said sample is BAL or BAS. Furthermore, the sample may come, for example, from cell cultures, environmental samples such as water, soil, surface or food samples.
The term “support”, as used in the present invention, relates to any solid material whereto the components of the invention, in particular the antibodies, the haptens or the bioconjugates of the invention, are physically bound, thus being immobilized. Any of a wide variety of solid supports may be used in the immunoassays of the present invention. The suitable materials for the solid support are synthetic such as polystyrene, polyvinyl chloride, polyamide or other synthetic polymers, natural polymers such as cellulose, and derivative natural polymers such as cellulose acetate or nitrocellulose, and glass, especially glass fibres. The support may take the form of spheres, sticks, tubes and microassay or microtiter plates. Structures similar to sheets such as strips of paper, small plates and membranes are also suitable. The surface of the supports may be permeable and impermeable for aqueous solutions. Additional inorganic solid supports suitable for their use in the present invention include, but are not limited to, silicon, crystal, quartz, ceramic, metals and their oxides, silica, silicates, silicides, nitrides, amorphous silicon carbide and any other material suitable for microfabrication or microlithography. Additional organic solid supports suitable for their use in the present invention include, without limitation, polymers such as polyimide, acrylate, polymethylmetacrylate, polystyrene or nitrocellulose.

### BRIEF DESCRIPTION OF THE FIGURES

FIG. 1. Concentration of quinolones measured in culture broth samples at 8 hours of growth from bacterial isolates classified as acute (1-5) or chronic (7-11) according to clinically diagnosed patients.

The following examples serve to illustrate the invention and should not be considered, in any case, as limiting of the scope thereof.

### EXAMPLES

### Example 1. Chemistry

### General Procedures and Equipment

The chemicals used in the synthesis of the haptens were obtained from Aldrich Chemical Co. (Milwaukee, Wis., USA), Sigma Chemical Co. (St. Louis, Mo., USA) or Acros Organics B.V.B.A. (Morris Plains, N.J., USA). Thin-layer chromatography (TLC) was performed on 0.25 mm, pre-coated silica gel 60 F254 aluminium sheets (Merck, Darmstadt, Germany). ¹H and ¹³C NMR spectra were obtained with a Varian Mercury-400 spectrometer (400 MHz ¹H and 101 MHz for ¹³C). Liquid chromatography/electrospray ionization/mass spectrometry (LC/ESI/MS) was performed in a Waters (Milford, Mass., USA) model composed by an Acquity UPLC system directly interfaced to a Micromass LCT Premier XE MS system equipped with an ESI LockSpray source for monitoring positive and negative ions. Data were processed with MassLynx (V 4.1) software (Waters).

### Example 1.1. Preparation of Intermediates

### Intermediates II

### 4-(2-((tert-butyldimethylsilyl)oxy)ethyl)aniline (1)

Imidazol (4.7 g, 0.07 mol) was added to a mixture of 2-(4-aminophenyl)ethanol (8.0 g, 0.05 mol) and TBSCl (10.5 g, 0.07 mol) in DMF (115 mL). The reaction was stirred at room temperature for 4 h. Afterwards, water and ethyl acetate were added to the reaction and the mixture was extracted with ethyl acetate 3 times. The combined organic layers were washed with water, brine, dried over MgSO₄ and evaporated under reduced pressure. Crude product was purified by silica flash chromatography using as eluent AcOEt/Hexane 9:1. Pure aniline 1 was obtained 11.3 g, yield 90% as orange oil.

¹H NMR (400 MHz, CDCl₃) δ 6.99 (d, J=8.5 Hz, 2H), 6.62 (d, J=8.5 Hz, 2H), 3.74 (t, J=7.3 Hz, 2H), 2.72 (t, J=7.3 Hz, 2H), 0.88 (s, 9H), 0.00 (s, 6H). ¹³C NMR (101 MHz, CDCl₃) δ 144.64, 130.05, 129.25, 115.28, 65.12, 38.95, 26.11, 18.52, −5.20. HRMS: m/z (ES+) for C₁₄H₂₆NOSi [(M+H)⁺] calculated 252.1784 found 252.1788 (+1.6 ppm).

### Methyl 3-(4-aminophenyl)propanoate (2)

Thionyl Chloride (0.75 ml, 0.01 mol) was added to a stirred ice cooled MeOH solution (3 ml). The mixture was left stirring 10 min and 3-(4-aminophenyl) propanoic acid was slowly added (0.5 g, 0,003 mol). The mixture was stirred 16 h under inert atmosphere at room temperature. After completion, the mixture was evaporated under reduced pressure and NaHCO₃ std. solution was added. The aqueous phase was extracted 3 times using ethyl acetate. Organic layers were combined, washed with brine, dried over Na₂SO₄ and evaporated under reduced pressure. The crude product was washed with hexane and evaporated to obtain aniline 2 (0.525 g, 0.0029 mol, yield 97%) as a single product.

¹H NMR (400 MHz, CD₃OD) δ 6.94 (d, J=8.3 Hz, 2H), 6.66 (d, J=8.3 Hz, 2H), 3.62 (s, 3H), 2.78 (t, J=7.6 Hz, 2H), 2.55 (t, J=7.6 Hz, 2H). ¹³C NMR (101 MHz, CD₃OD) δ 175.37, 146.69, 131.64, 129.88, 116.93, 51.96, 37.12, 31.26. HRMS: m/z (ES+) for C₁₀H₁₃NO₂ [(M+H)⁺] calculated 180.1025 found 180.1028 (+1.7 ppm).

### Intermediate III

### 5-(1-hydroxyoctylidene)-2,2-dimethyl-1,3-dioxane-4,6-dione (3)

Meldrum's acid (10 g, 0.069 mol) was dissolved in anhydrous DCM (127.6 mL) and cooled to 0° C. under N₂ atmosphere. After cooling, pyridine (11.18 mL, 0.14 mol) was added followed by dropwise addition of octanoyl chloride (12.414 g, 0.076 mol). The reaction mixture was stirred 1 h at 0° C. then it was left stirring at RT. Reaction progress was monitored by TLC until completion. Reaction mixture was washed with 5% HCl solution. The organic layer was then washed with distilled water before being dried over anhydrous MgSO₄, filtered and concentrated under reduced pressure to yield compound 3 as an orange oil (18.59 g, quantitative yield) which was used in the next step without further purification.

¹H NMR (400 MHz, CDCl₃) δ 3.06 (t, J=7.8 Hz, 2H), 1.73 (s, 6H), 1.69 (m, 2H), 1.45-1.20 (m, 8H), 0.87 (t, J=6.8 Hz, 3H). ¹³C NMR (101 MHz, CDCl₃) δ 198.47, 170.72, 160.33, 104.89, 91.37, 35.89, 31.75, 29.46, 29.03, 26.94, 26.29, 22.72, 14.19. HRMS: m/z (ES−) for C₁₄H₂₁O₅ [(M−H)⁻] calculated 269.1389 found 269.1388 (−0.4 ppm).

### Intermediate IV

### Methyl 3-oxodecanoate (4)

5-(1-hydroxyoctylidene)-2,2-dimethyl-1,3-dioxane-4,6-dione 3 (18.5 g, 0.068 mol) was dissolved in MeOH (84 mL) and heated at reflux for 5 h. The reaction was allowed to cool, and the solvent was removed under reduced pressure yielding the crude product as an orange oil. Purification was performed using silica flash chromatography Hex/Et₂O 8:2. Compound β-ketoester 4 was obtained as a yellow oil (8.85 g, 0.044 mol, yield 76%).

¹H NMR (400 MHz, CDCl₃) δ 3.72 (s, 3H, O—CH₃), 3.43 (s, 2H, OC—CH₂—CO), 2.51 (t, J=7.4 Hz, 2H, CH₂), 1.57 (quin., 2H, CH₂), 1.34-1.17 (m, 8H, 4×CH₂), 0.86 (t, J=6.7 Hz, 3H, CH₃). ¹³C NMR (101 MHz, CDCl₃) δ 202.94, 167.80, 52.41, 49.12, 43.19, 31.75, 29.12, 29.07, 23.58, 22.70, 14.16. HRMS: m/z (ES−) for C₁₁H₂₀O₃ [(M−H)⁻] calculated 199.1334 found 119.1331 (−2.5 ppm)

### Intermediates V

### Methyl 3-((4-(3-(tert-butyldimethylsilyl)propyl)phenyl)amino)dec-2-enoate (5)

To a solution of compound 4 (7.0 g, 35 mmol) in dry hexane (100 mL) was added aniline 1 (9.7 g, 38 mmol) and p-toluene sulfonic acid (0.12 g, 0.7 mmol). The reaction mixture was heated at reflux under a N₂ atmosphere for 16 h. It was allowed to cool down and the reaction mixture was evaporated under reduced pressure, obtaining the crude product 5 (14.5 g, 34 mmol, yield 97%) as an pale orange oil. The product was used in the next step without further purification.

¹H NMR (400 MHz, CDCl₃) δ 10.22 (s, 1H), 7.16 (d, J=8.3 Hz, 2H), 7.00 (d, J=8.3 Hz, 2H), 4.70 (s, 1H), 3.80 (t, J=6.8 Hz, 2H), 3.68 (s, 3H), 2.79 (t, J=6.8 Hz, 2H), 2.25 (t, J=7.7 Hz, 2H), 1.48-1.35 (m, 2H), 1.38-1.09 (m, 8H), 0.85-0.91 (m, 12H), −0.05 (s, 6H). ¹³C NMR (101 MHz, CDCl₃) δ 171.19, 164.30, 137.34, 136.82, 129.99, 125.31, 84.09, 64.46, 50.37, 39.11, 32.33, 31.74, 29.21, 28.98, 28.16, 26.05, 22.72, 18.47, 14.19, −5.28. HRMS: m/z (ES+) for C₂₅H₄₄NO₃Si [(M+H)⁺] calculated 434.3090 found 434.3087 (−0.7 ppm).

### Methyl 3-((4-(3-methoxy-3-oxopropyl)phenyl)amino)dec-2-enoate (6)

To a solution of compound 4 (1.0 g, 5 mmol) in dry toluene (15 mL) was added aniline 2 (0.98 g, 5.5 mmol) and p-toluene sulfonic acid (0.017 g, 0.1 mmol). The reaction mixture was heated at 85° C. under a N₂ atmosphere for 16 h. It was allowed to cool down and the reaction mixture was evaporated under reduced pressure, yielding the crude product as a pale orange oil. The crude product 6 (1.947 g) was used in the next step without further purification (purity by NMR about 70%).

¹H NMR (400 MHz, CDCl₃) δ 10.22 (s, 1H), δ 7.15 (d, J=8.3 Hz, 2H), 7.00 (d, J=8.3 Hz, 2H), 4.70 (s, 1H), 3.68 (s, 3H), 3.67 (s, 3H), 2.94 (t, J=7.8 Hz, 2H), 2.63 (t, J=7.8 Hz, 2H), 2.25 (t, J=8.0 Hz, 2H), 1.40 (quin., 2H), 1.33-1.13 (m, 8H), 0.84 (t, J=6.9 Hz, 3H). ¹³C NMR (101 MHz, CDCl₃) δ 173.38, 171.16, 164.09, 129.07, 128.94, 125.43, 120.49, 84.38, 51.79, 50.39, 35.74, 32.32, 31.70, 30.48, 29.17, 29.14, 28.94, 22.69, 14.17. HRMS: m/z (ES+) for C₂₁H₃₁NO₄ [(M+H)⁺] calcd 362.2331 found 362.2346 (+4.1 ppm).

### Intermediates VI

### 6-(2-((tert-butyldimethylsilyl)oxy)ethyl)-2-heptylquinolin-4(1H)-one (7)

Compound κ (13.2 g, 30.4 mmol) was added dropwise to refluxing diphenyl ether (250 ml) at 270° C. and maintained for 2 h. Once the reaction cooled to RT, a silica preparative column was used in order to eliminate the diphenyl ether, using hexane as eluent. The crude product was re-absorbed in the same silica using DCM and evaporating under reduced pressure. Afterwards, the product was purified by flash column chromatography using a concentration gradient of eluent from Hexane/AcOEt 4:6 to 3:7. Quinolone 7 was obtained as off-white solid (6.23 g, 15.5 mmol, yield 51%).

¹H NMR (400 MHz, CDCl₃) δ 11.99 (s, 1H), 8.18 (d, J=1.9 Hz, 1H), 7.65 (d, J=8.5 Hz, 1H), 7.47 (dd, J=8.5, 1.9 Hz, 1H), 6.20 (s, 1H), 3.82 (t, J=7.0 Hz, 2H), 2.91 (t, J=7.0 Hz, 2H), 2.67 (t, J=7.8 Hz, 2H), 1.76-1.64 (m, 2H), 1.38-1.09 (m, 8H), 0.90-0.74 (m, 12H), −0.04 (s, 6H, H17 and H18). ¹³C NMR (101 MHz, CDCl₃) δ 178.89, 154.88, 139.34, 134.92, 133.57, 125.05, 124.93, 118.40, 108.04, 64.56, 39.43, 34.50, 31.80, 29.33, 29.22, 29.13, 26.05, 22.71, 18.43, 14.16, −5.24. HRMS: m/z (ES−) for C₂₄H₃₈NO₂Si [(M−H)⁻] calculated 400.2672 found 400.2678 (+1.5 ppm).

### Methyl 3-(2-heptyl-4-oxo-1,4-dihydroquinolin-6-yl)propanoate (8)

Following an analogous procedure to that described for compound 7, but using ester 6, compound 8 was obtained as a pale-brown solid after purification by flash chromatography column using DCM with 2% MeOH (0.927 g, 2.8 mmol, yield 75%).

1H NMR (400 MHz, CDCl₃) δ 11.64 (s, 1H), δ 8.18 (d, J=2.0 Hz, 1H), 7.62 (d, J=8.5 Hz, 1H), 7.45 (dd, J=8.5, 2.0 Hz, 1H), 6.20 (s, 1H), 3.64 (s, 3H), 3.04 (t, J=7.8 Hz, 2H), 2.71-2.62 (m, 4H), 1.76-1.61 (m, 2H), 1.34-1.13 (m, 8H), 0.81 (t, J=6.8 Hz, 3H). 13C NMR (101 MHz, CDCl3) δ 178.78, 173.28, 154.74, 139.23, 136.16, 132.70, 125.13, 124.26, 118.65, 108.27, 51.79, 35.73, 34.50, 31.79, 30.73, 29.28, 29.13, 29.11, 22.70, 14.15. HRMS: m/z (ES−) for C20H26NO3 [(M−H)−] calculated 328.1913 found 328.1904 (−2.8 ppm).

### Example 1.2. Preparation of Haptens I

### Haptens 1a. Preparation of 2-heptyl-6-(2-mercaptoethyl)quinolin-4(1H)-one (I-1)

### i) 6-(2-bromoethyl)-2-heptylquinolin-4(1H)-one (9)

To a solution of quinolone 7 (0.15 g, 0.37 mmol) in 5 mL of anhydrous DCM, a solution of boron tribromide in DCM 1M was slowly added under inert atmosphere. The mixture was left under refluxe at 45-50° C. overnight. The reaction mixture was evaporated under reduced pressure. H₂O was added and extracted 3 times with AcOEt. Combined organic layers were washed with NaHCO3std, brine, dried over MgSO4 and evaporated under vacuum. The crude product was purified by silica column chromatography using DCM with 2% MeOH as eluent. It was obtained pure compound 9 (0.13 g, 0.36 mmol, yield 96%).

¹H NMR (400 MHz, CD₃OD) δ 8.07 (d, J=2.0 Hz, 1H), 7.59 (dd, J=8.6, 2.0 Hz, 1H), 7.54 (d, J=8.5 Hz, 1H), 6.21 (s, 1H), 3.67 (t, J=7.2 Hz, 2H), 3.27 (t, J=7.2 Hz, 2H), 2.69 (t, J=7.8 Hz, 2H), 1.74 (p, J=7.4 Hz, 2H), 1.45-1.25 (m, 8H), 0.89 (t, J=6.7 Hz, 3H). ¹³C NMR (101 MHz, CD₃OD) δ 180.40, 156.96, 140.49, 136.57, 134.32, 125.52, 125.45, 119.31, 108.83, 39.78, 34.97, 33.68, 32.85, 30.18, 30.17, 30.08, 23.65, 14.38. HRMS: m/z (ES−) for C₁₈H₂₃BrNO [(M−H)⁻] calculated 348.0963 found 348.0963 (+0.0 ppm).

### ii) S-(2-(2-heptyl-4-oxo-1,4-dihydroquinolin-6-yl)ethyl) ethanethioate (10)

A solution of bromoderivative 9 (70 mg, 0.20 mmol) and potassium thioacetate (22.8 mg, 0.20 mmol) in 2.0 mL of anhydrous DMF was stirred during 1 h. The reaction was diluted with AcOEt and washed 3 times with H2O, brine, dried over Na₂SO₄ and evaporated under reduced pressure to obtain pure compound 10 (69 mg, 0.20 mmol, quantitative yield), used in the next step without further purification.

¹H NMR (400 MHz, CD₃OD) δ 8.05 (d, J=2.0 Hz, 1H), 7.59 (dd, J=8.6, 2.0 Hz, 1H), 7.53 (d, J=8.5 Hz, 1H), 6.21 (s, 1H), 3.16 (t, J=7.1 Hz, 2H), 2.98 (t, J=7.1 Hz, 2H), 2.70 (t, J=7.6 Hz, 2H), 2.30 (s, 3H), 1.75 (p, J=7.5 Hz, 2H), 1.45-1.26 (m, 8H), 0.89 (t, J=6.9 Hz, 3H). ¹³C NMR (101 MHz, CD₃OD) δ 197.12, 180.40, 156.87, 140.34, 137.50, 134.32, 125.41, 125.24, 119.31, 108.79, 36.49, 34.97, 32.85, 31.25, 30.51, 30.18, 30.08, 23.65, 14.38. HRMS: m/z (ES−) for C₂₀H₂₆NO₂S [(M−H)⁻] calculated 344.1684 found 344.1682 (−0.6 ppm).

### iii) 2-Heptyl-6-(2-mercaptoethyl)quinolin-4(1H)-one (I-1)

Protected thiol 10 (69 mg, 0.2 mmol) and KOH (11.2 mg, 0.2 mmol) were dissolved under inert atmosphere in 1 ml of anhydrous and degassed MeOH. The mixture was stirred during 1 h at RT. The reaction was acidified until pH 6-7 with degassed HCl 1M, diluted with water and extracted 3 times with AcOEt, washed with brine, dried over Na₂SO₄ and evaporated under reduced pressure to obtain the crude product as a pale yellow solid. Eventually, the crude product was purified by crystallization using hexane/AcOEt to obtain compound I-1 (49 mg, 0.16 mmol, yield 81%).

¹H NMR (400 MHz, CDCl₃) δ 12.17 (s, 1H), 8.17 (d, J=2.0 Hz, 1H), 7.74 (d, J=8.5 Hz, 1H), 7.44 (dd, J=8.5, 2.0 Hz, 1H), 6.26 (s, 1H), 3.00 (t, J=7.3 Hz, 2H), 2.79 (q, J=7.5 Hz, 2H), 2.71 (t, J=7.8 Hz, 2H), 1.72 (p, J=7.6 Hz, 2H), 1.37-1.12 (m, 8H), 0.80 (t, J=6.8 Hz, 3H).

¹³C NMR (101 MHz, CDCl₃) δ 178.54, 155.37, 139.44, 135.62, 133.00, 124.87, 124.70, 118.89, 108.08, 39.94, 34.49, 31.79, 29.30, 29.23, 29.12, 26.09, 22.71, 14.18. HRMS: m/z (ES−) for C₁₈H₂₄NOS [(M−H)⁻] calculated 302.1579 found 302.1575 (−1.0 ppm).

### Haptens 1b. Preparation of 3-(2-heptyl-3-hydroxy-4-oxo-1,4-dihydroquinolin-6-yl)propanoic acid (I-2)

### i) 3-(3-Formyl-2-heptyl-4-oxo-1,4-dihydroquinolin-6-yl)propanoic acid (11)

Ester 8 (700 mg, 2.1 mmol), hexamine (601.7 mg, 4.3 mmol) and p-TsOH·H2O (453.6 mg, 2.4 mmol, 1.1 equiv) were dissolved in glacial acetic acid (54 ml). The mixture was heated at reflux for 3 h under a nitrogen atmosphere. After cooling, 6 M HCl (13 ml) was added and heating was continued at 115° C. for 1 h. The mixture was allowed to cool, diluted with water, and extracted with ethyl acetate. The combined organic fractions were washed with brine, dried over MgSO4, and concentrated under reduced pressure. Ethanol was used to recover some precipitated product during filtration, evaporated under vacuo and mixed with crude product for purification. The crude was purified by column chromatography on silica flash chromatography using DCM with 6% MeOH and 0.5% glacial acetic acid to obtain acid 11 as an off-white solid (480 mg, 1.4 mmol, yield 66%).

1H NMR (400 MHz, DMSO-d6) δ 12.13 (s, 1H), 10.38 (s, 1H), 7.97 (d, J=2.0 Hz, 1H), 7.62 (dd, J=8.4, 2.0 Hz, 1H), 7.51 (d, J=8.4 Hz, 1H), 3.03 (t, J=7.6 Hz, 2H), 2.94 (t, J=7.4 Hz, 2H), 2.58 (t, J=7.4 Hz, 2H), 1.60 (quin., 2H), 1.41-1.21 (m, 8H), 0.86 (t, J=6.7 Hz, 3H). 13C NMR (101 MHz, DMSO-d6) δ 190.81, 177.98, 173.58, 159.61, 137.98, 137.59, 133.64, 126.12, 123.84, 118.74, 113.23, 48.59, 35.12, 31.54, 31.12, 29.98, 28.98, 28.85, 28.34, 22.04, 13.92. HRMS: m/z (ES−) for C20H24NO4 [(M−H)−] calculated 344.1705 found 344.1708 (+0.9 ppm).

### ii) 3-(2-Heptyl-3-hydroxy-4-oxo-1,4-dihydroquinolin-6-yl)propanoic acid (I-2)

Aqueous hydrogen peroxide (1.05 M, 1.0 ml, 1.0 mmol) and aqueous sodium hydroxide (1.08 M, 1.78 ml, 1.9 mmol) were added to a solution of acid 11 (0.300 g, 0.9 mmol) in ethanol (4.3 ml) under nitrogen atmosphere. The mixture was stirred overnight at room temperature. After completion, the reaction mixture was evaporated under reduced pressure. The crude was purified by flash column chromatography using DCM with 4% MeOH and 0.5% glacial acetic acid. Quinolone 1-2 was obtained (135 mg, 0.4 mmol, yield 47%).

1H NMR (400 MHz, DMSO-d6) δ 11.38 (s, 1H), δ 7.90 (d, J=1.6 Hz, 1H), 7.45 (d, J=8.6 Hz, 1H), 7.43 (dd, J=8.6, 1.6 Hz, 1H), 2.91 (t, J=7.5 Hz, 2H), 2.71 (t, J=7.5 Hz, 2H), 2.57 (t, J=7.5 Hz, 2H), 1.64 (quin., 2H), 1.37-1.18 (m, 8H), 0.84 (t, J=6.6 Hz, 3H). 13C NMR (101 MHz, DMSO-d6) δ 173.69, 168.59, 137.76, 135.95, 135.25, 134.14, 130.86, 122.96, 122.07, 117.86, 35.33, 31.19, 30.04, 28.77, 28.47, 28.12, 27.82, 22.06, 13.93. HRMS: m/z (ES−) for C19H25NO4 [(M−H)−] calculated 330.1705 found 330.1699 (−1.8 ppm).

### Haptens 1c. Preparation of 6-(2-carboxyethyl)-2-heptyl-4-hydroxyquinoline N-oxide (I-3)

### i) Methyl 3-(4-((tert-butoxycarbonyl)oxy)-2-heptylquinolin-6-yl)propanoate (12)

Ester 8 (129 mg, 0.39 mmol) was dissolved in anhydrous THF (7 ml). Boc₂O (94 mg, 0.43 mmol) and a catalytic quantity of 4-(dimethylamino)pyridine (DMAP) (12 mg, 0.1 mmol) were added. Then, the mixture was heated at 60° C. under nitrogen atmosphere during 1 h 30. The mixture was left to cool to RT and concentrated under reduced pressure. The crude was purified by silica flash chromatography using AcOEt/Hex 8:2 as eluent, obtaining Boc protected compound 12 (148 mg, 0.34 mmol, yield 88%).

¹H NMR (400 MHz, CDCl₃) δ 7.97 (d, J=8.6 Hz, 1H), 7.76 (d, J=2.0 Hz, 1H), 7.55 (dd, J=8.6, 2.0 Hz, 1H), 7.25 (s, 1H), 3.68 (s, 3H), 3.13 (t, J=7.8 Hz, 2H), 2.94 (t, J=7.8 Hz, 2H), 2.72 (t, J=7.8 Hz, 2H), 1.85-1.73 (m, 2H), 1.62 (s, 9H), 1.46-1.22 (m, 8H), 0.87 (t, J=6.8 Hz, 3H). ¹³C NMR (101 MHz, CDCl₃) δ 173.25, 163.68, 154.20, 150.66, 148.62, 138.51, 131.15, 129.14, 120.75, 119.74, 112.28, 84.72, 51.84, 39.64, 35.71, 31.90, 31.21, 30.07, 29.64, 29.30, 27.83, 22.77, 14.22. HRMS: m/z (ES+) for C₂₅H₃₆NO₅ [(M+H)⁺] calculated 430.2593 found 430.2579 (−3.3 ppm).

### ii) 4-((tert-butoxycarbonyl)oxy)-2-heptyl-6-(3-methoxy-3-oxopropyl)quinoline N-oxide (13)

Compound 12 (138 mg, 0.32 mmol) was dissolved in anhydrous DCM (4 ml) and cooled at 4° C. Afterwards, mCPBA (83 mg, 0.48 mmol) was added and the mixture was stirred at 4° C. under nitrogen atmosphere during 4 h. Once the starting material was consumed, more DCM was added, and the solution was washed 3 times with NaHCO₃std. The organic phase was concentrated under reduced pressure and it was obtained, without further purification, pure N-oxide 13 (132 mg, 0.30 mmol, yield 92%) as a yellow oil.

1H NMR (400 MHz, CDCl₃) δ 8.70 (d, J=9.0 Hz, 1H), 7.77 (d, J=1.9 Hz, 1H), 7.63 (dd, J=9.0, 1.9 Hz, 1H), 7.31 (s, 1H), 3.66 (s, 3H), 3.13 (m, 4H), 2.72 (t, J=7.8 Hz, 2H), 1.88-1.75 (m, 2H), 1.61 (s, 9H), 1.49-1.22 (m, 8H), 0.87 (t, J=6.8 Hz, 3H). 13C NMR (101 MHz, CDCl3) δ 172.93, 150.56, 149.57, 144.13, 141.33, 140.92, 132.08, 122.97, 120.69, 120.59, 113.61, 85.22, 51.90, 35.36, 31.87, 31.81, 30.95, 29.69, 29.19, 27.80, 26.24, 22.76, 14.20. HRMS: m/z (ES+) for C25H36NO6 [(M+H)+] calculated 446.2542 found 446.2539 (−0.7 ppm).

### iii) 6-(2-Carboxyethyl)-2-heptyl-4-hydroxyquinoline N-oxide (I-3)

N-oxide 13 (125 mg, 0.28 mmol) was dissolved in a degassed solution of KOH 5 M in EtOH (2.5 ml). The mixture was stirred at RT under nitrogen atmosphere during 1 h. Afterwards, H₂O was added, and the mixture was left stirring 30 min. The mixture was acidified with HCl cc until pH=1-2 when a white solid precipitated. It was filtered and dried to obtain the crude product. Quinolone N-oxide 1-3 (72 mg, 0.22 mmol, yield 77%) was obtained after crystallization in EtOH/H2O 4:1.

1H NMR (400 MHz, CD3OD) δ 8.11 (d, J=2.0 Hz, 1H), 8.04 (d, J=8.8 Hz, 1H), 7.73 (dd, J=8.8, 2.0 Hz, 1H), 6.35 (s, 1H), 3.09 (t, J=7.6 Hz, 2H), 2.92 (t, J=7.6 Hz, 2H), 2.70 (t, J=7.6 Hz, 2H), 1.83-1.73 (m, 2H), 1.53-1.24 (m, 8H), 0.91 (t, J=6.9 Hz, 3H). 13C NMR (101 MHz, CD3OD) δ 176.27, 155.85, 140.65, 139.67, 134.59, 125.20, 124.52, 117.17, 107.29, 36.34, 32.88, 32.48, 31.56, 30.43, 30.11, 28.82, 23.69, 14.39. HRMS: m/z (ES−) for C19H2404 [(M−H)−] calculated 330.1705 found 330.1710 (+1.5 ppm).

### Example 2. Immunochemistry

### General Procedures and Equipment

The reagents used were obtained from Aldrich Chemical Co. (Milwaukee, Wis., USA) and from Sigma Chemical Co. (St. Louis, Mo., USA). Purification of conjugates was carried out in ÄKTA Prime Plus using 2 HiTrap desalting columns both from GE Healthcare (Chicago, Ill., USA) or either by dialysis using Spectra/Por membranes from Spectrumlabs (Piraeus, Greece, EU) with molecular weight cut-off of 12-14 kDa. The matrix-assisted laser desorption ionization time-of-flight mass spectrometer (MALDI-TOF-MS) was a Bruker autoflex III Smartbeam spectrometer (Billerica, Mass.). The pH and the conductivity of all buffers and solutions were measured with a pH-meter pH 540 GLP and a conductimeter LF 340, respectively (WTW, Weilheim, Germany). Polystyrene microtiter plates were purchased from Nunc (Maxisorp, Roskilde, Denmark). Dilution plates were purchased from Nirco (Barbera del Valles, Spain). Washing steps were performed on a Biotek ELx465 (Biotek Inc.). Absorbances were read on a SpectramaxPlus (Molecular Devices, Sunnyvale, Calif., USA) at a single wavelength mode (450 nm). The competitive curves were analysed with a four-parameter logistic equation using the software GraphPad Prism 7.0 (GraphPad Software Inc., San Diego, Calif., USA).

### Buffers

Unless otherwise indicated, phosphate buffer saline (PBS) corresponds to 10 mM phosphate buffer in 0.8% saline solution (pH 7.5). Coating buffer is a 50 mM bicarbonate-carbonate buffer (pH 9.6). PBST is PBS with 0.05% Tween 20 (pH 7.5). Citrate buffer corresponds to 40 mM sodium citrate (pH 5.5).

The substrate solution contains 0.01% of 3,3′,5,5′-tetramethylbenzidine (TMB) and 0,004% H₂O₂ prepared in citrate buffer. Borate buffer is 0.2 M sodium borate/boric acid (pH 8.7). All buffers were prepared using ultra-pure Milli-Q® water with a resistivity between 16-18 MΩ cm.

### Analysis of Hapten Density

Hapten densities of protein conjugates were estimated by means of MALDI-TOF-MS comparing molecular weight of natural proteins with that of conjugates. MALDI experiments were conducted by deposition of 2 μL of the matrix solution (10 mg mL⁻¹ of sinapinic acid in MeCN/H₂O 70:30, 0.1% HCOOH) in the MALDI plate and after drying, 2 μL of the purified sample diluted ½ with MeCN 0.2% HCOOH are added and allowed to dry. Finally, 2 μL of the matrix solution are added over the mixture mentioned above and after drying the resulting spot analyzed by MALDI-TOF. Hapten densities were calculated through the equation: [MW(conjugate)−MW(native protein)]/[MW(hapten)−MW(lost atoms)].

### Example 2.1. Immunogens and Coating Antigens Preparation

### Conjugation of I-1 Hapten

The conjugation procedure was carried out in parallel over 25 mg of HCH (Horseshoe Crab Hemocyanin) or 25 mg of BSA (Bovine Serum Albumine). Each protein was dissolved in 4.5 ml of Borax/Borate buffer pH=8.7. Afterwards 12.5 mg (44 μmol) of succinimidyl iodoacetate (SIA) were dissolved in 1 mL of anhydrous DMF. Over each protein solution, dropwise additions (5×100 μL) of SIA were performed. The reactions were stirred 3.5 h at RT and left overnight at 4° C. without agitation to obtain iodoacetate-BSA and iodoacetate-HCH solutions, which were purified by AKTA using 2 HiTrap desalting column and eluting with Borax/Borate buffer.

8.0 mg (26 μmol) of I-1 hapten were dissolved in 1.7 mL of anhydrous DMF. Afterwards, 850 μL of I-1 solution were added dropwise to each activated protein solution and the mixtures were stirred 4 h at RT and left overnight at 4° C. without agitation. Finally, each immunoreactive agent obtained was purified by dialysis in PBS 0.5 mM (5×5 L) and Milli-Q water (1×5 L) and lyophilized.

### Conjugates Obtained:

2-((2-(2-heptyl-4-oxo-1,4-dihydroquinolin-6-yl)ethyl)thio)acetamide-HCH (1-4)
2-((2-(2-heptyl-4-oxo-1,4-dihydroquinolin-6-yl)ethyl)thio)acetamide-BSA (1-5)

### Conjugation of I-2 and I-3 Haptens

The conjugation procedure was carried out in parallel over a solution of 5 mg of KLH (Keyhole Limpet Hemocyanin) or a solution of 5 mg of BSA (Bovine Serum Albumine) in PBS 10 mM.

A solution of 2.62 μL (11 μmop of tri-n-butylamine and 1.56 μL (12 μmop isopropyl cloroformiate was added to a solution of the corresponding hapten, 1-2 or 1-3 to activate the carboxylic acid, (10 μmop dissolved in 400 μL of anhydrous DMF. The mixture was left stirring 15 min at 4° C. and 30 min at RT. Then 200 μL of the reaction mixture were added over each protein solution and the mixture was left 2 h stirring at RT and left overnight at 4° C. without agitation. Each immunoreactive agent obtained was purified by dialysis in PBS 0.5 mM (5×5 L) and Milli-Q water (1×5 L) and lyophilized.

### Conjugates Obtained:

3-(2-heptyl-3-hydroxy-4-oxo-1,4-dihydroquinolin-6-yl)propanamide-KLH (I-6)
3-(2-heptyl-3-hydroxy-4-oxo-1,4-dihydroquinolin-6-yl)propanamide-BSA (I-7)
3-(2-heptyl-1-hydroxy-4-oxo-1,4-dihydroquinolin-6-yl)propanamide-KLH (I-8)
3-(2-heptyl-1-hydroxy-4-oxo-1,4-dihydroquinolin-6-yl)propanamide-BSA (I-9)

### Conjugate Density

TABLE 2
Quantity of bioconjugates produced and hapten density of
BSA conjugates calculated from MALDI-TOF analysis.
HCH or KLH conjugates cannot be analyzed by MALDI-TOF,
therefore the degree of conjugation was evaluated by comparison
with a paralel conjugation protocol with BSA conjugates.

| | Quantity (mg) | Yield (%) | N° of residues |
|---|---|---|---|
| SIA-BSA | — | — | 22 |
| I-4 | 18.4 | 74 | — |
| I-5 | 18.73 | 75 | 13 |
| I-6 | 5.29 | 105 | — |
| I-7 | 5.95 | 119 | 17 |
| I-8 | 6.68 | 133 | — |
| I-9 | 3.38 | 67.6 | 21 |

### Example 2.2. Antibody Production

Antibodies were obtained by immunizing female New Zealand white rabbits with the corresponding immunogen, namely 1-4, 1-6 or 1-8.

The protocol used for the production of antibodies was conducted in accordance with the institutional guidelines under a license from the local government (DAAM 7463) and approved by the Institutional Animal Care and Use Committee at the CID-CSIC.

The antisera (As) obtained by immunizing the animals were named as:

| Immunogen | Antisera | Coating antigen |
|---|---|---|
| I-4 | As382, As383 and As384 | I-5 |
| I-6 | As385, As386 and As387 | I-7 |
| I-8 | As388, As389 and As390 | I-9 |

The antibody titer was assessed during the immunization process through non-competitive indirect ELISA. Microtiter plates were coated with a fixed concentration of the homologous competitor conjugate (1 mg mL⁻¹) and the avidity of the produced antibodies was measured by preparing serial dilutions of the corresponding As. The animals were exsanguinated after 6 immunizations, and the final blood was collected in vacutainer tubes provided with a serum separation gel. Antisera were obtained by centrifugation at 4° C. for 10 min at 10 000 rpm, then stored at −80° C. in the presence of preservative 0.02% sodium azide.

### Example 2.3. Non-Competitive Indirect 2D ELISA

Non-competitive indirect ELISA were carried out to establish the concentrations of a homologous coating antigen (CA) and As dilutions used in competitive assays. For this purpose, concentrations of BSA conjugates (I-5, I-7, I-9) ranging from 5 μg/ml to 5 ng/ml and As dilutions from 1/1000 to 1/1024000 were assessed. The experimental procedure is fully detailed in the next section. However, in this type of assay no analyte is present, therefore the total volume of As dilution added per well is 100 μL.

Competitive assay conditions were selected at 70% signal saturation giving approximately 1-1.2 units of absorbance.

### Example 2.4. Competitive Indirect ELISA

The competitive assay was carried out in a 96 well Maxisorp flat-bottom plates, coated using 100 μL of a BSA conjugate solution in coating buffer pH=9.6. Then, plates were covered with adhesive plate sealer and incubated overnight at 4° C. The day after, plates were washed with PBST (4×300 μL) using the platewasher Biotek ELx405 HT. Sequentially, 50 μL of the corresponding sample solution, containing the analyte HHQ, PQS or HQNO (2 μM to 0.13 nM) or MH medium were added, followed by 50 μL addition of a fixed As dilution and left without agitation 30 min at RT. After another washing step, a 1/6000 dilution of goat AntiRabbit IgG-HRP in PBST was added and incubated 30 min at RT. After a final washing, 100 μL of a substrate solution was added and left 30 min at RT in the dark. Once the time was consumed, the enzymatic reaction was stopped by adding 50 μl of H₂SO₄ 4M solution and the absorbances read at 450 nm. Absorbance data were plotted and analyzed using GraphPad software. The standard calibration curve was fitted to a four-parameter equation according to the following formula: y=B+(A−B)/[1−(x/C)^{D}], where A is the maximum absorbance, B is the minimum absorbance, C is the concentration producing 50% of the maximal absorbance, and D is the slope at the inflection point of the sigmoid curve. Unless otherwise indicated, the data presented correspond to the average of at least two well replicates.

TABLE 3
Parameters of competitive ELISA assays for detection of A) HHQ,
B) PQS and C) HQNO. The data shown correspond to the average of 3
different days suing at least 2 well/replicates per concentration

| A) | | PBST | MH diluted 1/5 |
|---|---|---|---|
| | As382 dil. | 1/32000 | 1/64000 |
| | I-7 (μg/mL) | 0.313 | 0.156 |
| | Aₘᵢₙ | 0.029 ± 0.005 | 0.012 ± 0.003 |
| | Aₘₐₓ | 1.187 ± 0.082 | 1.165 ± 0.081 |
| | Slope | −0.874 ± 0.114 | −0.704 ± 0.015 |
| | IC₅₀ | 4.593 ± 0.287 | 2.851 ± 0.296 |
| | Dynamic | 0.894 ± 0.214 to | 0.448 ± 0.081 to |
| | Range | 22.797 ± 3.691 | 21.605 ± 4.736 |
| | LOD | 0.341 ± 0.132 | 0.167 ± 0.051 |
| | R² | 0.995 ± 0.003 | 0.998 ± 0.002 |
| | | PBS + 0.01% | |
| B) | | Tween + 0.1 mM EDTA | MH diluted 1/10 |
| | As385 dil. | 1/48000 | 1/64000 |
| | I-5 (μg/mL) | 0.039 | 0.039 |
| | Aₘᵢₙ | 0.030 ± 0.006 | 0.019 ± 0.006 |
| | Aₘₐₓ | 1.445 ± 0.035 | 1.107 ± 0.025 |
| | Slope | −0.723 ± 0.006 | −0.734 ± 0.110 |
| | IC₅₀ | 3.872 ± 0.529 | 6.051 ± 0.155 |
| | Dynamic | 0.528 ± 0.043 to | 0.973 ± 0.250 to |
| | Range | 24.365 ± 3.182 | 36.157 ± 6.197 |
| | LOD | 0.169 ± 0.012 | 0.362 ± 0.137 |
| | R² | 0.997 ± 0.002 | 0.995 ± 0.003 |
| C) | | PBS at pH = 6.5 | MH diluted 1/5 |
| | As389 dil. | 1 | 1/16000 |
| | I-5 (μg/mL) | 0.250 | 0.250 |
| | Aₘᵢₙ | 0.091 ± 0.036 | 0.091 ± 0.020 |
| | Aₘₐₓ | 1.083 ± 0.059 | 0.854 ± 0.009 |
| | Slope | −0.754 ± 0.037 | −0.720 ± 0.064 |
| | IC₅₀ | 4.204 ± 0.858 | 2.708 ± 0.035 |
| | Dynamic | 0.723 ± 0.181 to | 0.413 ± 0.102 to |
| | Range | 26.707 ± 0.958 | 17.044 ± 1.076 |
| | LOD | 0.266 ± 0.087 | 0.147 ± 0.053 |
| | R² | 0.987 ± 0.013 | 0.990 ± 0.004 |

### Example 2.5. ELISA Evaluation

### Physicochemical Parameters Optimization

Performance of the assays was evaluated through the modification of different physicochemical parameters in the competition step. The assessed parameters were: competence time, incubation time, pH, ionic strength, presence of a surfactant (% Tween 20), solubility with addition of organic solvents or cation complexation by EDTA.

TABLE 4
Physicochemical parameters selected after optimization.
The parameters improving the features of
the assay were assessed separately and in conjunction.

| | Assay in buffer | | |
|---|---|---|---|
| | HHQ | PQS | HQNO |
| As dilution | 1/32000 | 1/32000 | 1/16000 |
| | (As382) | (As385) | (As389) |
| [Competitor] (μg/mL) | 0.313 | 0.039 | 0.250 |
| pH | 7.5 | 7.5 | 6.5 |
| Conductivity (mS/cm) | 15 | 15 | 15 |
| Tween 20 (%) | 0.05 | 0.01 | 0.05 |
| Competition time (min) | 30 | 30 | 30 |
| Preincubation time (min) | 0 | 0 | 0 |
| Organic solvent (%) | 0 | 0 | 0 |

### Specificity or Cross Reactivity Studies

Regarding specificity studies, it was followed the experimental procedure for indirect competitive ELISA described above. It was assessed the avidity of As versus other quinolone effector molecules of the pqs system from P. aeruginosa. Each assay was run using HHQ, PQS and HQNO as analyte. Cross reactivity was calculated through the equation: CR (%)=IC₅₀(Cross reactant)/IC₅₀(Analyte)×100.

TABLE 5
Percentages of Cross Reactivity (CR) calculated for HHQ, PQS and HQNO using
the corresponding As under the conditions of the three developed assays.

| | As382 (HHQ) | | As385 (PQS) | | As389 (HQNO) | |
|---|---|---|---|---|---|---|
| Analyte | IC50 (nM) | C.R. (%) | IC50 (nM) | C.R. (%) | IC50 (nM) | C.R. (%) |
| HHQ | 2.67 | 100.00 | 27.19 | 13.31 | 26.35 | 36.24 |
| PQS | 37.27 | 7.16 | 3.62 | 100.00 | 176.60 | 5.41 |
| HQNO | 85.97 | 3.11 | 236.20 | 1.53 | 9.55 | 100.00 |

### Example 2.6. Clinical Isolates

### Matrix Effect Study

Culture broth Mueller-Hinton was diluted 1:2, 1:5, 1:10, 1:20 and used to run the standard calibration curve. Subsequently, the dilution providing the best ELISA parameters was selected and the conditions of CA and As dilution adjusted.

### Accuracy Studies

Blind spiked samples using diluted MH culture broth were prepared, measured and interpolated in the standard curve mentioned above. The experiment was repeated three different days and the final accuracy results are expressed as mean of all replicates.

### Reproducibility Study

The assay was run three different days and three times within the same day.

### Growth Curves

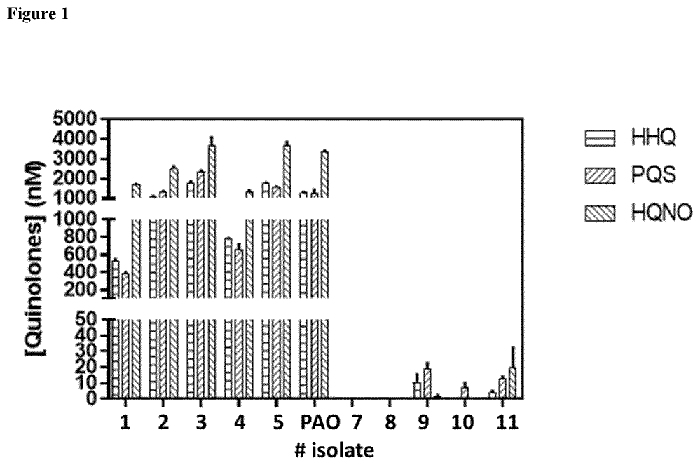

Clinical Pseudomonas aeruginosa isolates coming from patients diagnosed with acute or chronic respiratory airways infection were grown in MH culture broth 8 h at 37° C. In addition, a reference P. aeruginosa strain (PAO) was also grown under the same conditions. Then, aliquots were taken, centrifuged and analysed using the developed immunochemical assays. The results enclosed in FIG. 1 suggest a clear difference in the QS production profile between both types of isolates. Therefore, it would be possible to differentiate and stratify patients depending on the pqs quorum sensing system molecular footprint.

It has been demonstrated the potential of the immunochemical tools presented here for differentiation of both types of infections. The three studied quinolones are promising biomarkers for diagnostic of infections caused by P. aeruginosa. Moreover, the study of QS could provide much more information about type of infection or disease state.

1. A compound characterized by the Formula I, 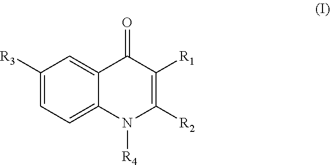
wherein:
R₁ is selected among H or OH; R₂ is selected among C3-C15 alkyl, C3-C15 alkenyl or C3-C15 alkynyl; R₃ is (CH₂)ₘ—R₅; m is a whole number between 1 and 6; R₄ is selected among H or OH; and R₅ is selected among COOH, SH, NH₂, OH or PEG.

2. A compound, according to claim 1, selected from the group consisting of:

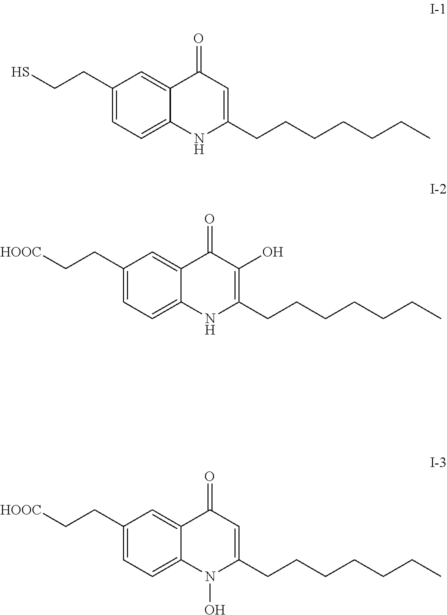 [SMILES: CCCCCCCc1cc(=O)c2cc(CCS)ccc2[nH]1 ; CCCCCCCc1[nH]c2ccc(CCC(=O)O)cc2c(=O)c1O ; CCCCCCCc1cc(=O)c2cc(CCC(=O)O)ccc2n1O]

3. Use of a compound, according to any of the claim 1 or 2, or any combination thereof, as a hapten.

4. Conjugate comprising at least a hapten according to any of the claim 1 or 2, in combination with a second component which confers antigenicity to the conjugate, characterized in that the R₃ of Formula I forms a covalent bond with the second component.

5. Conjugate, according to claim 4, wherein the second component is a carrier protein, or a fragment thereof, selected from the group comprising: horseshoe crab hemocyanin (HCH), bovine serum albumine (BSA) or keyhole limpet hemocyanin (KLH).

6. Method for producing a conjugate, according to any of the claim 4 or 5, which comprises creating a covalent bond, directly or through a cross-linking agent, between the carrier protein and at least one hapten according to any of the claim 1 or 2, wherein the covalent bond is formed between the carrier protein and the R₃ of the hapten.

7. Use of the conjugate according to any of the claim 4 or 5 for producing antibodies.

8. Antibody characterized in that it specifically recognizes a conjugate as defined in claim 4 or 5, or antiserum comprising said antibody.

9. In vitro method for detecting and/or quantifying at least a quinolone selected from the group: 2-heptyl-4-quinolone (HHQ), 2-heptyl-3-hydroxy-4-quinolone (PQS), and/or 2-heptyl-4-quinolone N-oxide (HQNO), in a biological sample, which comprises the use of an antibody or antiserum as defined in claim 8.

10. In vitro method, according to claim 9, wherein the detection and/or quantification is carried out by an immunochemical technique, preferably the immunochemical technique is an ELISA.

11. In vitro method, according to any of the claim 9 or 10, which comprises:
a) immobilizing a conjugate defined in any of claim 4 or 5 on a solid support,
b) eliminating the non-immobilized conjugate,
c) adding the sample to be analysed and a first antibody defined in claim 9 in the solid support of section a) and incubating,
d) eliminating the first antibody not bound to the conjugate,
e) adding a second antibody conjugated with a detectable labelling agent, said second antibody recognizing the first antibody and incubating,
f) eliminating the second antibody not bound to the first antibody, and
g) detecting and/or quantifying the complex obtained according to section e) with a composition containing a chromogenic, fluorogenic and/or chemiluminescent indicator substrate.

12. In vitro method, according to any of claims 9 to 11, wherein the sample is obtained from a subject who may have an infection caused by Pseudomonas aeruginosa.

13. In vitro method, according to any of claims 9 to 12, wherein the sample is selected from the group comprising: sputum, bronchoaspirate, bronchoalveolar lavage, blood, serum and/or plasma.

14. Kit for the detection and/or quantification a quinolone selected from the group: 2-heptyl-4-quinolone (HHQ), 2-heptyl-3-hydroxy-4-quinolone (PQS), and/or 2-heptyl-4-quinolone N-oxide (HQNO), characterized in that it comprises at least one antibody defined in claim 8, or a conjugate defined in any of claim 4 or 5, or any combination thereof.
